(19) United States
(12) Patent Application Publication
(10) Pub. No.: US 2019/0228867 A1
(43) Pub. Date: Jul. 25, 2019

(54) PATIENT-CENTRIC MEDICAL INFORMATION SYSTEM INCLUDING MOBILE APPLICATION, SCORING MODEL AND PHYSICIAN PORTAL

(71) Applicant: SYMPTELLIGENCE MEDICAL INFORMATICS, LLC, New York, NY (US)

(72) Inventors: Stuart K.J. Smyth, Newmarket, NH (US); Jerry G. Blaivas, New York, NY (US)

(21) Appl. No.: 15/311,294

(22) PCT Filed: May 18, 2015
(86) PCT No.: PCT/US15/31450

§ 371 Date: Nov. 26, 2018

(51) Int. Cl. G16H 80/00; G16H 10/60; G16H 10/20; G06F 3/0482; G06F 3/0483; G06F 16/2457; G06F 16/248

(52) U.S. Cl. CPC G16H 80/00; G16H 10/60; G16H 10/20; G06F 16/248; G06F 3/0483; G06F 16/24578; G06F 16/24573; G06F 3/0482

(57) ABSTRACT

Methods, systems and computer readable media for patient-centric medical information systems (and methods and computer readable media) including a mobile application, a scoring model and a physician portal are described.

### RELATED APPLICATIONS

This application claims the benefit of U.S. Provisional Application No. 61/936,879, entitled “MEDICAL INFORMATICS SYSTEM” and filed on May 16, 2014, which is incorporated herein by reference in its entirety.

### TECHNICAL FIELD

Some implementations relate generally to medical information systems and, more particularly, to patient-centric medical information systems including a mobile application and a physician portal.

### BACKGROUND

With the growing confines resulting from healthcare reform and other changes, physicians are often pressured to see more patients in less time for less reimbursement, which may force physicians to essentially treat patient populations by a rubric. One problem in clinical practice may be that truly individualized care often requires collecting and analyzing clinical data that only the patient can provide.

Due to the changing healthcare delivery platform, individualized treatment may be undermined by the often time consuming and cumbersome nature of collecting necessary clinical data. This problem may be exacerbated by an enormous administrative burden that may become too expensive for the physician. As a result, sufficient data may not be collected and patients may be treated empirically (e.g., by trial and error). This trial and error approach can lead to inaccurate diagnoses, improper treatment for patients, and ultimately a greater financial burden on the system.

A need may exist for a system such as that disclosed herein that can empower patients to track and understand their own health, and in doing so, to fill the gaps that cause such disruption to the system. Some implementations were conceived in light of the above-mentioned problems and limitations, among other things.

### SUMMARY

Before a patient even arrives at the physician's office, he/she is instructed to download an implementation of the disclosed mobile application and track his/her own symptoms (health behavior) for a 24-hour period. The software intelligently creates a clinical summary that can be seamlessly incorporated in the electronic medical record (EMR). When the doctor sees the patient for the first time, the clinical picture is already painted and discussion between the doctor and patient can focus on filling in specifics; therefore, instead of trying to sift through an enormous clinical picture to pinpoint symptoms and causes, the doctor is provided with all of the necessary clinical information he would hope to collect in the first two patient visits.

The current healthcare delivery model includes a physician collecting a general history in a limited time period and then advising a patient to try a medication and return depending on its efficacy. With an implementation of the disclosed medical information software, a doctor can begin with an individualized summary generated by the system, then spend time answering the patient's questions, and identifying and directly treating the underlying cause of the pathology. This system can save time for the physician, reduce or eliminate the cost and administrative burden of collecting and transcribing clinical data, and most importantly, provide the patient with an individualized, targeted, and correct diagnosis. An implementation can save the medical system the costs of multiple physician visits, improper prescriptions, and worse, unnecessary surgeries. Through the disclosed method, patients become part of the solution, helping doctors save time and money while ultimately making more informed clinical decisions that, in turn, saves the healthcare system money.

A software implementation can have similar appeal for academic institutions, pharmaceutical companies, and any physician interested in clinical research. Collecting clinical data often requires time, motivated patients, and a staff dedicated to gathering and amassing this information. In fact, most clinical trials must offer patients either financial incentives or the promise of a unique chance to try a new drug for their condition. Beyond this cost, pharmaceutical companies or any institution running a trial must employ recruiters to enroll patients and subsequently must pay research assistants to transcribe the data from the patients into a system that can be queried. The use of an implementation of the disclosed medical information system for trials and other clinical research can significantly reduce or eliminate this entire burden. All inclusion and exclusion criteria can be programmed within the mobile application so the cost of recruiters drops immensely (if not entirely). Further, the mobile application software/portal system accumulates data seamlessly within a database that can be pre-configured in the desired format for research queries.

Some implementations can include a Precertification Model for ACO's. For example, the disclosed system can be configured to work with global healthcare systems, such as fee for service basis, but can also be configured to operate in a pay for performance model (e.g., ACO Model which is rapidly advancing in the USA).

The incorporation of an implementation of the disclosed software system into a healthcare system can be beneficial for all stakeholders (e.g., patients, physicians, and/or ACO). The healthcare system may be seeking for cost control. Typically surgeries, and now some other procedures, require the physician to provide evidence that the treatment is appropriate for the patient. The burden may be on the physician or on the ACO itself. The payor (be it insurance companies, the government, or pharmacy benefit managers) will mandate that doctors prove treatment is necessary, prudent and appropriate if the doctor wants to be reimbursed for treating a particular symptom or condition. The disclosed medical information system is inherently configured to serve as a precertification tool and can transfer the information gathering task of precertification from the physician to the patient. The patient can record his/her symptoms using validated clinical tools to provide parameters that can categorize patients' symptoms. The motivation for the payor is clear and absolute: empirical treatment leads to more patient visits, prescription of drugs for a condition a patient doesn't have and even unnecessary surgeries. Mandating the precertification tool makes sense fiscally. Physicians will benefit from the time and money they will save along with the more nuanced care they will be able to provide. Patients, in turn, will get individualized care and start to better understand their own symptoms.

Some implementations can include a medical informatics and/or autoinformatics system for taking medical history, record keeping and informatics that can provide symptom specific questionnaires and diaries. Data can be entered on an application (e.g., on mobile device, tablet, laptop or desktop computer or the like) by the patient. Autoinformatics can include patients entering medical history or other medical related information themselves.

Some implementations do not require a connection to the internet. The app can summarize and categorize data for clinical use. In some implementations, the patient data is available for one or more of a personal electronic medical record (EMR), medical records (PDF or XML), diagnosis & treatment, monitoring outcomes, monitoring/preventing complications and adverse events and/or research.

### BRIEF DESCRIPTION OF THE DRAWINGS

FIG. 1 is a diagram of an example medical informatics system in accordance with at least one implementation.

FIG. 2 is a flowchart of an example medical informatics method in accordance with at least one implementation.

FIG. 3 is a diagram of an example computer system in accordance with at least one implementation.

FIGS. 4-10 show example user interface screens of a physician portal in accordance with at least one implementation.

FIGS. 11-24 show example user interface screens for a mobile application in accordance with at least one implementation.

### DETAILED DESCRIPTION

It will be appreciated that although the example implementations described herein are directed to urinary conditions, the medical informatics system can be applied to any suitable medical condition where use of a computerized medical informatics system may be desired.

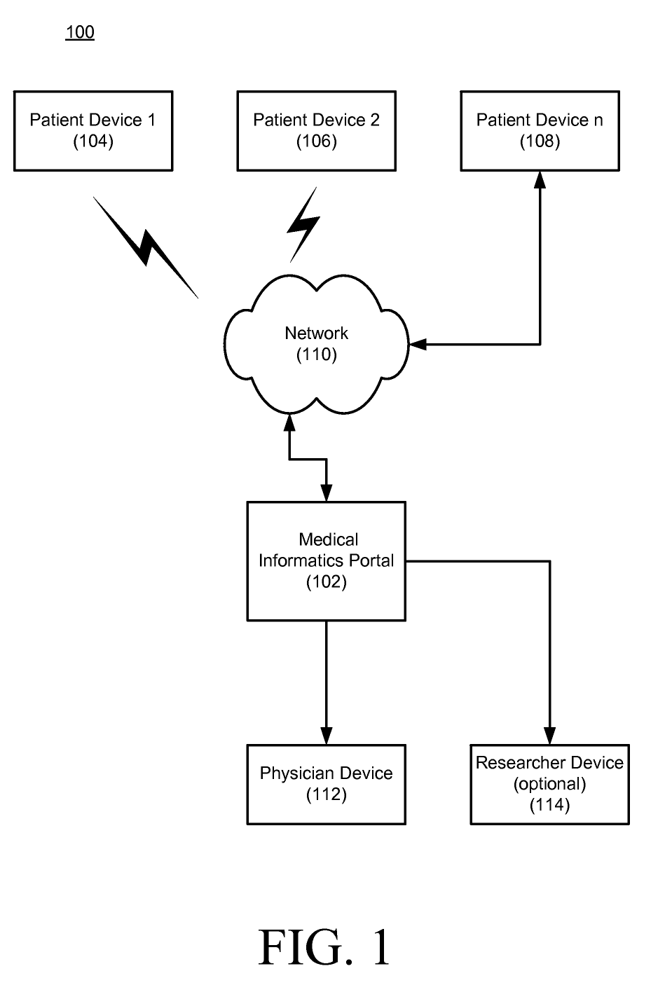

FIG. 1 is a diagram of an example medical informatics environment 100 in accordance with at least one implementation. In particular, the environment 100 can include a computerized medical informatics system 102. A plurality of user devices (104-108) can communicate with the computerized medical informatics system 102 via network 110. The computerized medical informatics system 102 can provide medical information for one or more patients to a physician device 112 and, optionally, to a researcher device 114.

The patient devices (104-108) can include a desktop computer, laptop computer, tablet computer, wireless device, tablet computer, wearable computer, electronic book reader, media player and/or the like. The network 110 can include a wired or wireless network (e.g., a WiFi network, the Internet, etc.). For example, one or more patient devices (e.g., 104 and 106) can communicate wirelessly with the network 110, while other patient devices (e.g., 108) can communicate via a wired interface.

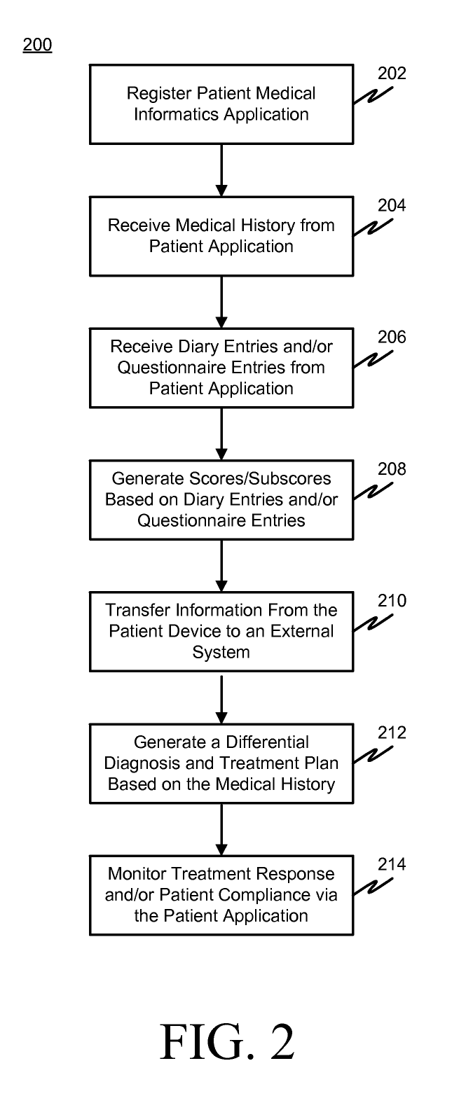

FIG. 2 is a flowchart of an example medical informatics method in accordance with at least one implementation. Processing begins at 202, where a patient (or other person) registers a medical informatics application. The registration process can include linking a patient's medical information to a specific identifier associated with the mobile application or mobile device. For example, a unique identifier of one or more mobile devices associated with the patient may be associated with the patient's medical records at a physician's office. Processing continues to 204.

At 204, medical history information is received from the patient. The medical history information can be received at a mobile device via text entry to a software application, voice recording, video recording, handwriting entry, gesture entry or the like. Processing continues to 206.

At 206, one or more patient diary entries and/or questionnaire responses are received by the mobile application. Processing continues to 208.

At 208, one or more scores and/or subscores are generated based on the diary entries and/or questionnaire responses. Processing continues to 210.

At 210, medical history, diary entries, questionnaire response(s), scores, and subscores are transferred from the mobile application to one or more external systems (e.g., a physician portal system and/or a research portal system). Processing continues to 212.

At 212 a differential diagnosis and/or treatment plan is automatically generated based on the medical history information. Processing continues to 214.

At 214, the system can monitor treatment response and/or patient compliance via the patient application. For example, medical diary entries in the patient application can be used to monitor treatment response and patient entries can also be used to monitor compliance with treatment plans.

It will be appreciated that 202-214 can be repeated in whole or in part in order to accomplish a contemplated medical informatics task.

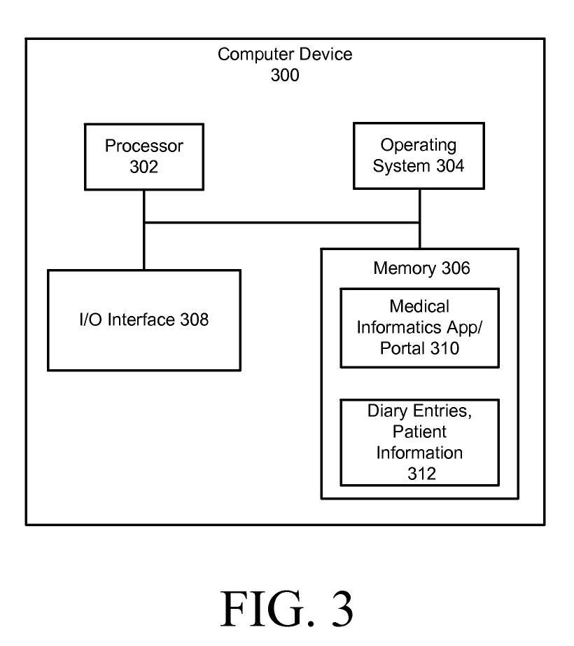

FIG. 3 is an example computer system 300 for computerized medical informatics in accordance with at least one implementation. The server device 300 includes a processor 302, an operating system 304, a memory 306 and an I/O interface 308. The memory 306 can include a medical informatics system application 310 and one or more medical informatics entries and/or patient information records 312.

In operation, the processor 302 may execute the application 310 stored in the memory 306. The application 310 can include software instructions that, when executed by the processor, cause the processor to perform operations for a computerized medical informatics system in accordance with the present disclosure (e.g., performing one or more of steps 202-210 described above).

The application program 310 can operate in conjunction with the one or more medical informatics entries and/or patient information records 312 and the operating system 304.

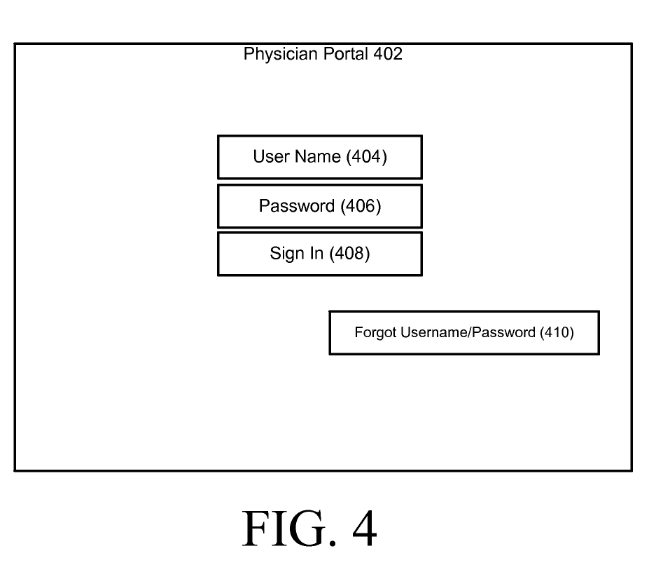

FIG. 4 is a diagram of an example physician portal 402 authentication user interface including a user interface element for receiving a user name (404) and a password (406). There are also elements for signing in (408) once the user name and password have been provided, and for following an alternate authentication route when a user name or password have been forgotten (410).

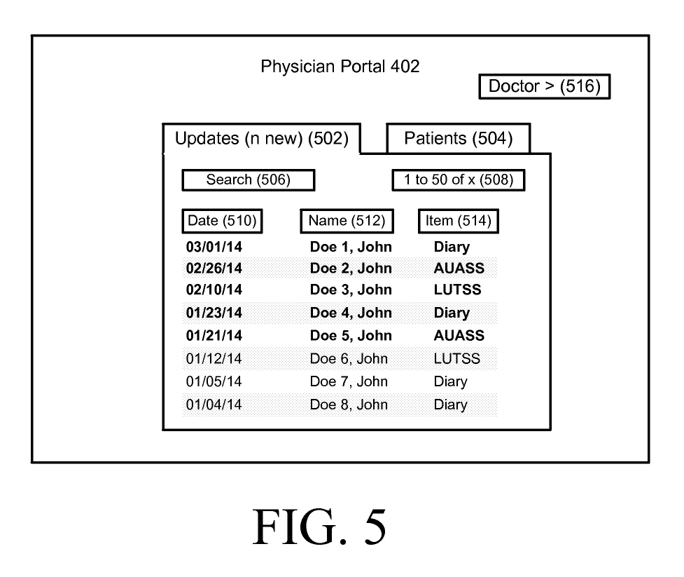

FIG. 5 shows an example physician portal 402 update tab 502 and patients tab 502. As shown in FIG. 5, the updates tab 502 has been selected and is showing update details including a search element 506, a number of updates being displayed 508, a date column 510, a name column 512 and an item column 514.

In operation, any updates which have been received and not viewed can be caused to be displayed as bold text (e.g., updates dating from Mar. 1, 2014-Jan. 21, 2014), while the viewed updates may be caused to be displayed as regular (e.g., not bold) text.

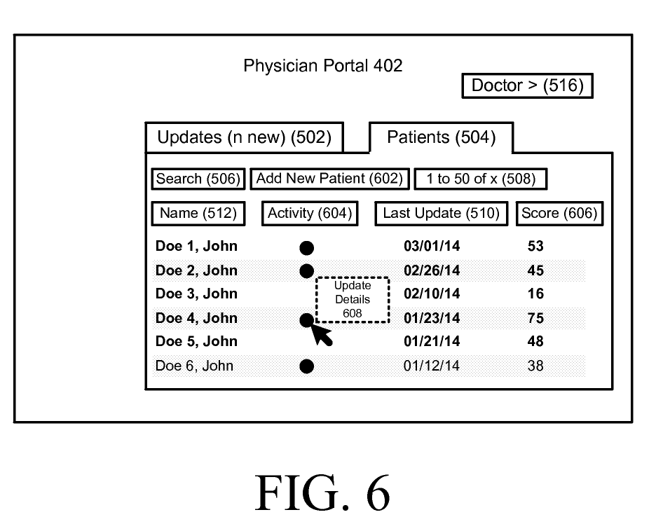

FIG. 6 shows an example physician portal 402 with the patients tab 504 selected. The patients tab 504 includes a search element 506, an add new patient element 602, and a number of records shown element 508. The patients tab also includes a name column 512, an activity column 604, a last update column 510 and a score column 606. By hovering over (or otherwise selecting) an activity element, an informational element (e.g., a tool tip) 608 can be caused to be displayed that shows addition update details relating to the activity.

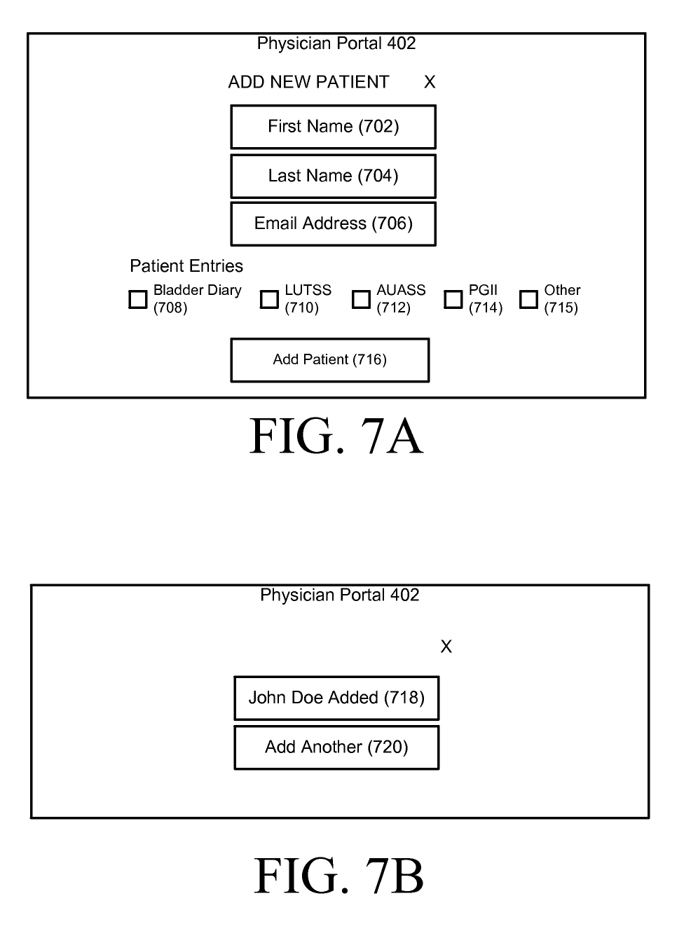

FIG. 7A shows a physician portal 402 add new patient screen. The add new patient screen can be caused to be displayed when the add new patient element (602) has been selected. The add new patient screen, when caused to be displayed, includes user interface elements for the following: first name 702, last name 704, email address 706, selection elements for receiving a selection of one or more patient diary entries requested by the physician for patient entry via the mobile application including bladder diary 708, LUTSS 710, AUASS 712, PGII 714, and other 715.

FIG. 7B shows a physician portal 402 new patient added confirmation screen with an element showing new patient added 718 and an element for adding another patient 720.

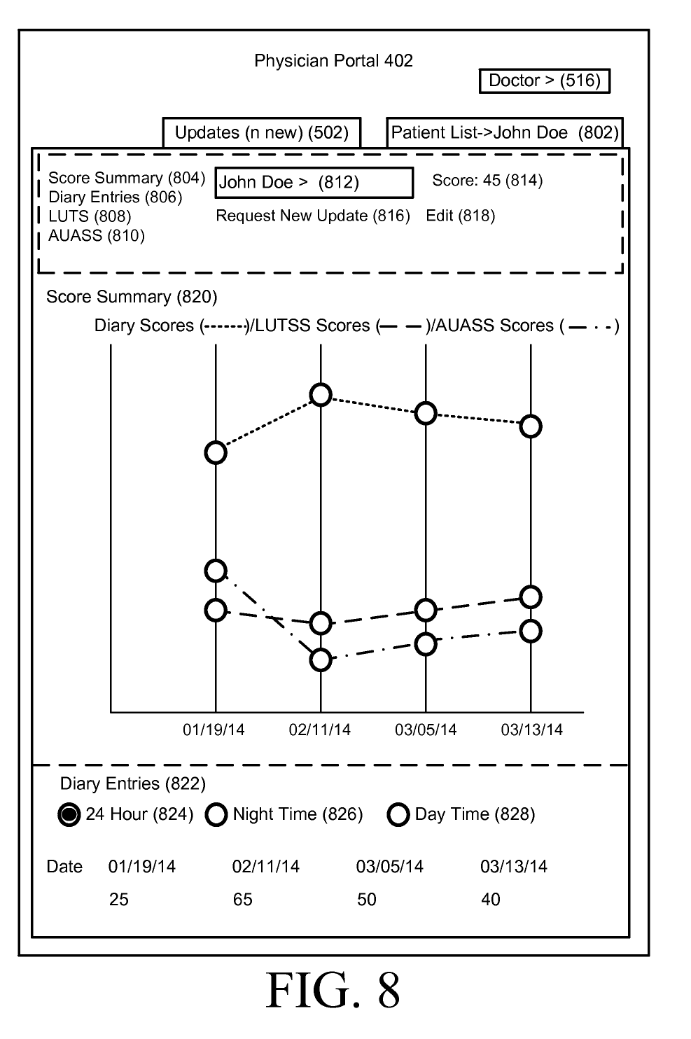

FIG. 8 shows a physician portal 402 patient detail screen that can be caused to be displayed when a selection of an individual patient is made from the list of patients shown in the patients tab. The patient detail screen includes an indication of the selected patient name 802 in the tab, a score summary 804 selection element, a diary entries selection element 806, an LUTSS score 808, an AUASS score element 810, a name element 812, a score element 814 (e.g., weScore), a request new update element 816 and an edit element 818.

A score summary section 820 of the patient detail screen shows one or more scores (e.g., diary scores, LUTSS scores and AUASS scores) for a given number of dates. The diary entries section 822 shows one or more selected diary entries (e.g., 24 hour 824, night time 826 and day time 828) for a given number of recent entries (e.g., the four most recent entries).

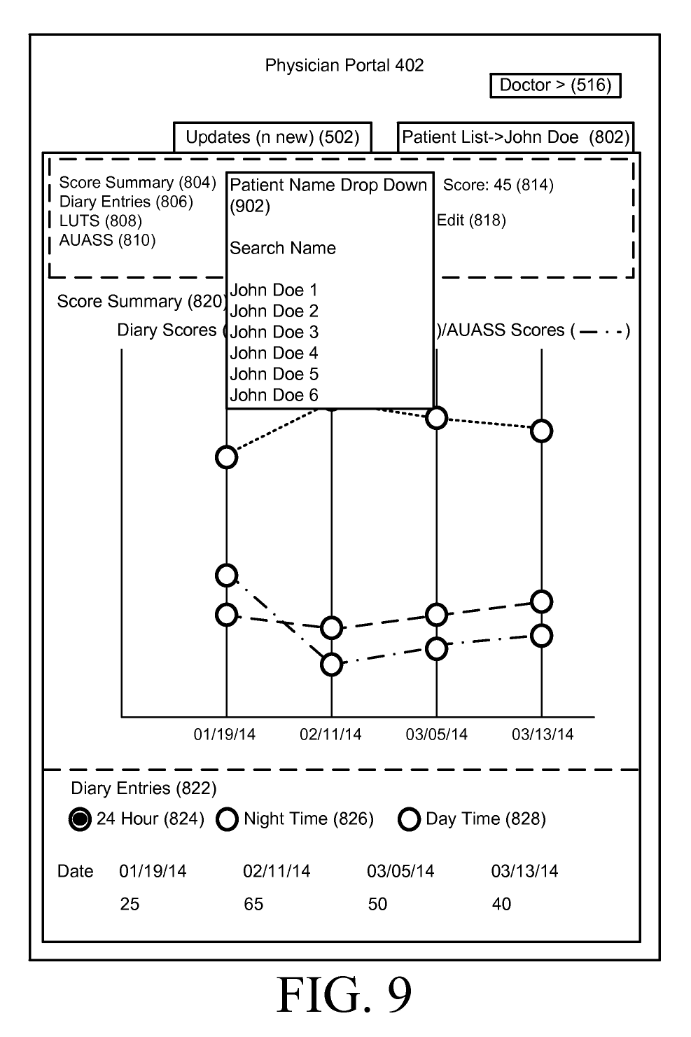

FIG. 9 shows a physician portal 402 patient detail screen with the patient name drop down element 902 activated and displaying a list of patient names and a search element for searching for a patient by name.

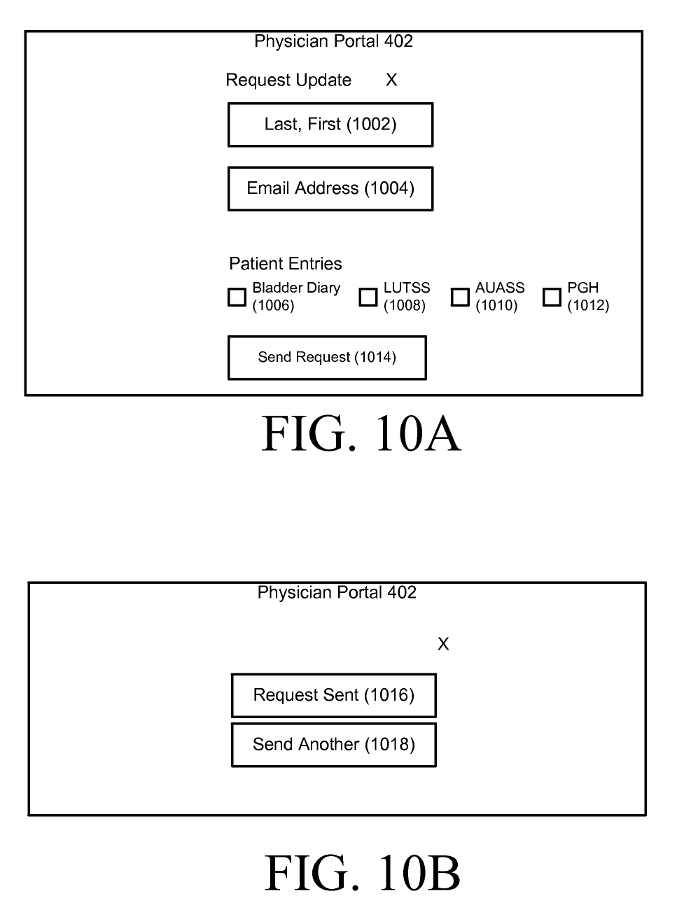

FIG. 10A shows a physician portal 402 request update screen that can be caused to be displayed when a request update user interface element (e.g., 816) is selected. The request update screen includes a name element 1002 for displaying or receiving entry or selection of a patient name and an email address element 1004 for displaying or receiving entry or selection of a patient email address. The request update screen also includes one or more selection elements for update type (e.g., 1006-1012) and a send request element 1012 that, when selected or activated, causes the update request to be sent to the mobile application on the patient device.

FIG. 10B shows a request sent confirmation screen having a request sent element 1016 and a send another element 1018 that, when selected causes a request update screen (e.g., as shown in FIG. 10A to be displayed).

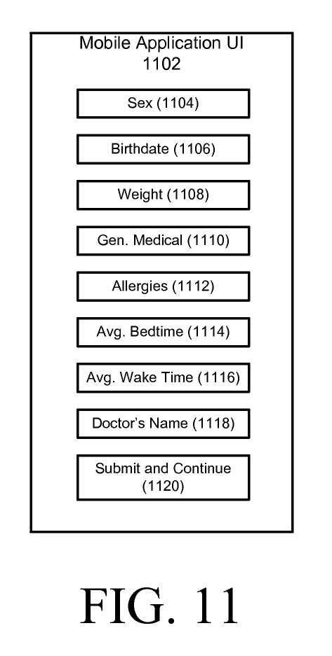

FIG. 11 shows a patient mobile application basic information user interface screen 1102 that can be caused to be displayed when a patient first uses the mobile application or when the patient selects a basic information element to edit the basic information already provided. The user interface 1102 includes elements for entering sex 1104, birth date 1106, weight 1108, general medical condition/information 1110, allergies 1112, average bedtime 1114, average wake time 1116, doctor's name 1118. The basic information screen 1102 also includes an element for submitting and continuation 1120.

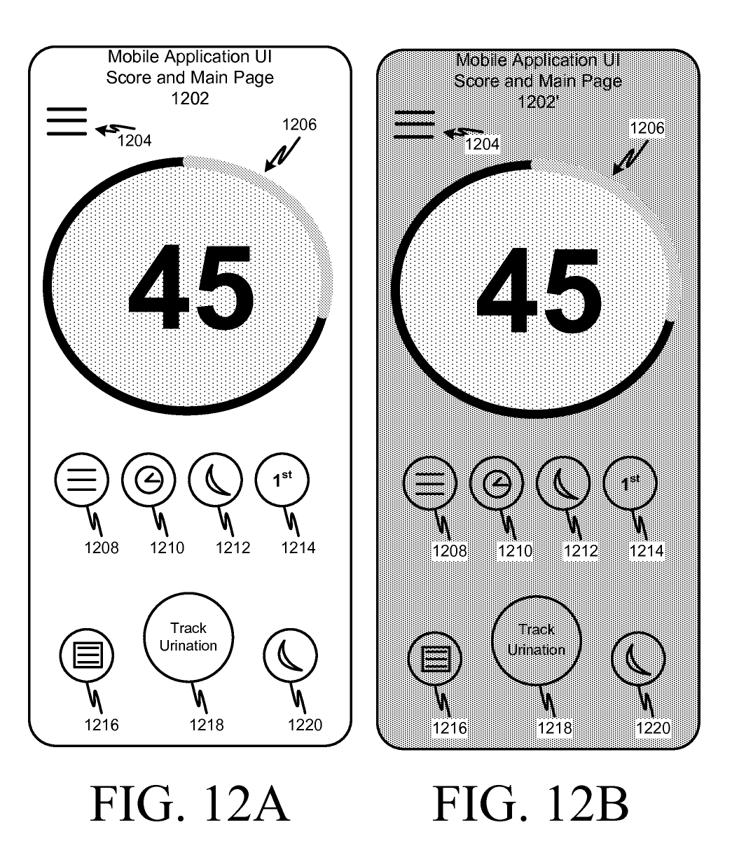

FIGS. 12A and 12B respectively show daytime and nighttime score and main page user interface screens. The main screen 1202 includes an options selection element 1204, a score indication 1206, a menu selection element 1208, a time confirm element 1210, a sleep time element 1212 and a first urination of the day element 1214. The interface also includes a diary element 1216, a record urination element 1218 and a sleep time element 1220.

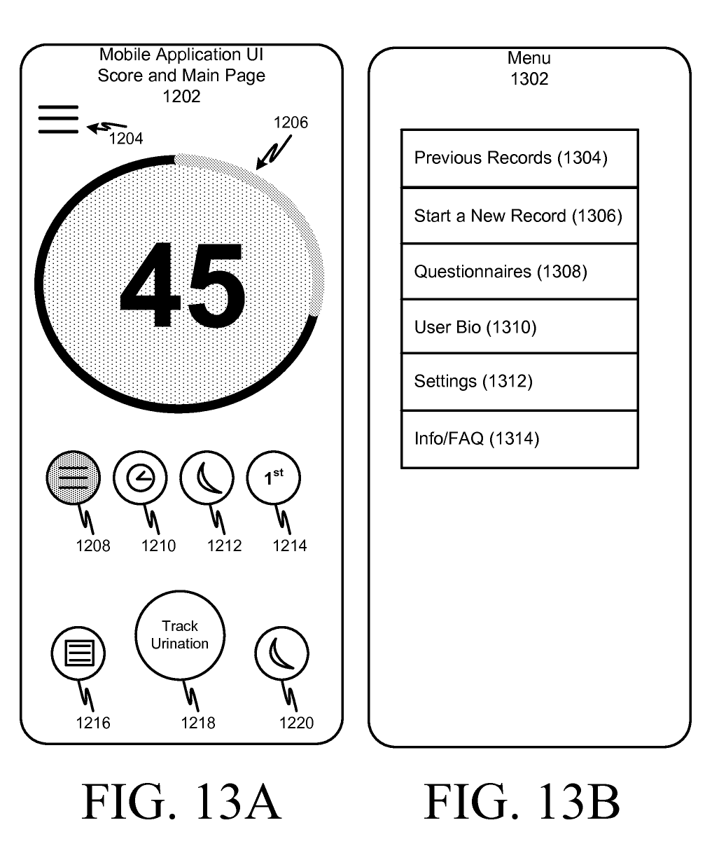

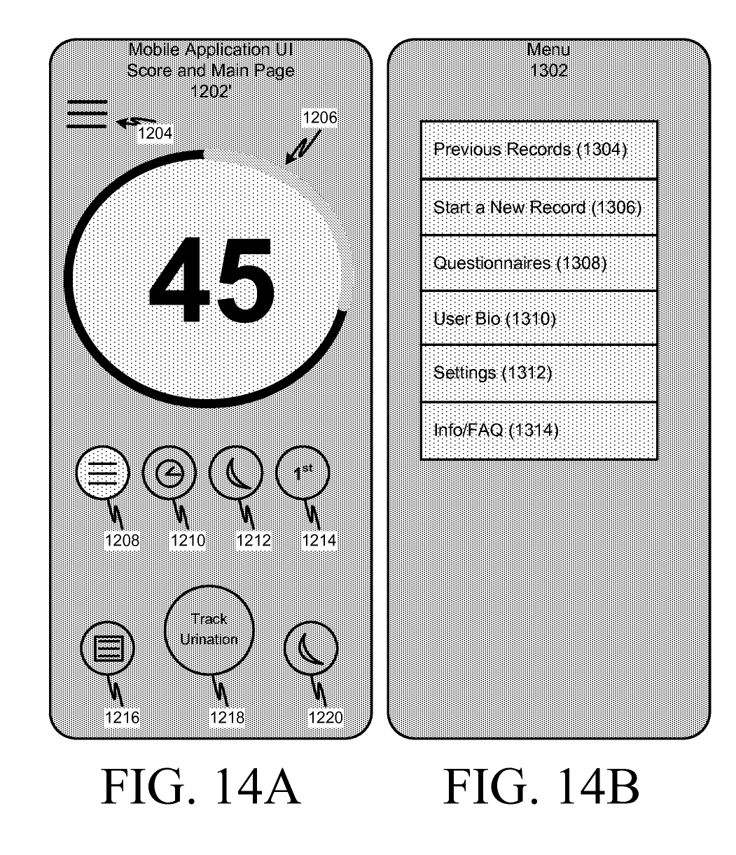

When the menu element 1208 is selected as shown in FIG. 13A, a menu interface is caused to be displayed as shown in FIG. 13B. The menu interface includes user interface controls for viewing previous records 1304, starting a new record 1306, user biographical information (or basic information) 1310, settings 1312 and an information or frequently asked questions (FAQ) control 1314. FIGS. 14A and 14B show nighttime versions of the main page 1202 and menu user interface 1302, respectively.

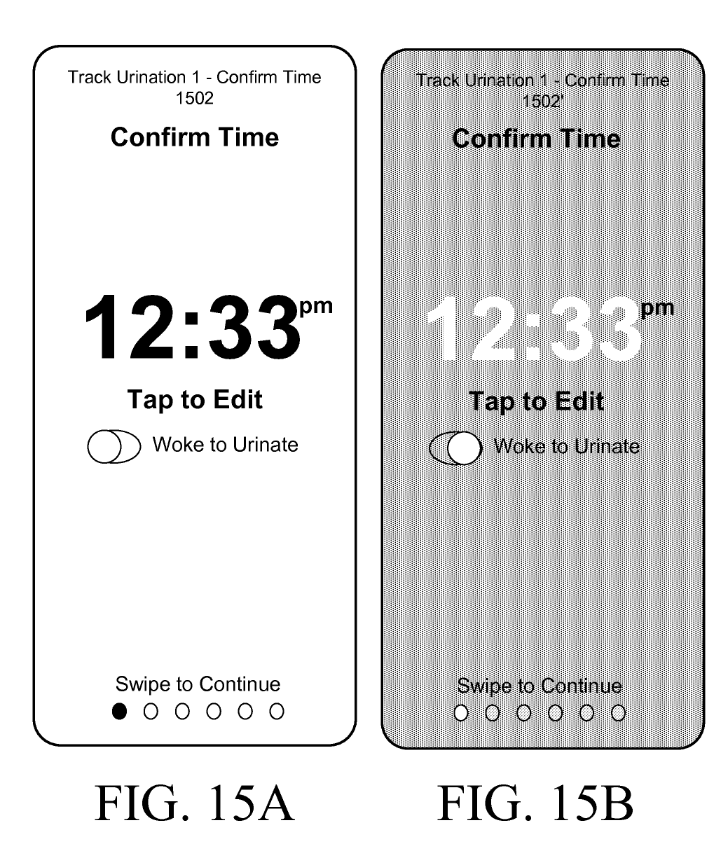

When the track (or record) urination control 1218 is selected, a sequence of two or more user interface screens is caused to be displayed (e.g., the user interface screens shown in FIGS. 15-20 and described below).

FIGS. 15A and 15B respectively show daytime and nighttime versions of a user interface 1502/1502′ for a first step of tracking urination via a mobile application. The first step includes confirming the time and indicating whether one woke to urinate. A gesture on a touch screen (e.g., “left swipe”) can be used to move to the next step of tracking urination.

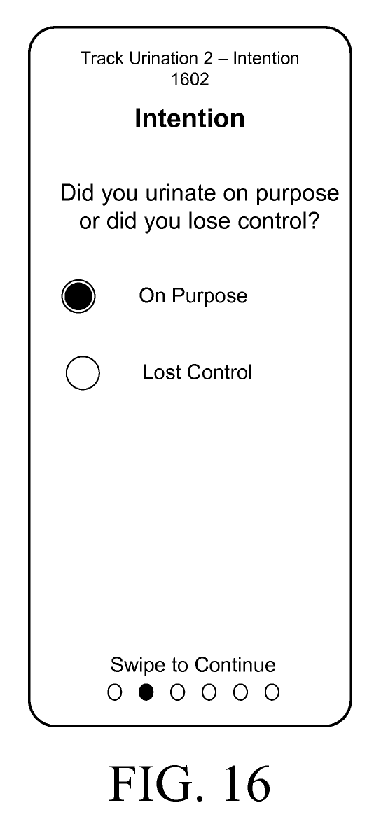

FIG. 16 shows a user interface 1602 for a second step of tracking urination in which a patient indicates whether urination was on purpose or due to lost control. A gesture on a touch screen (e.g., “left swipe”) can be used to move to the next step of tracking urination.

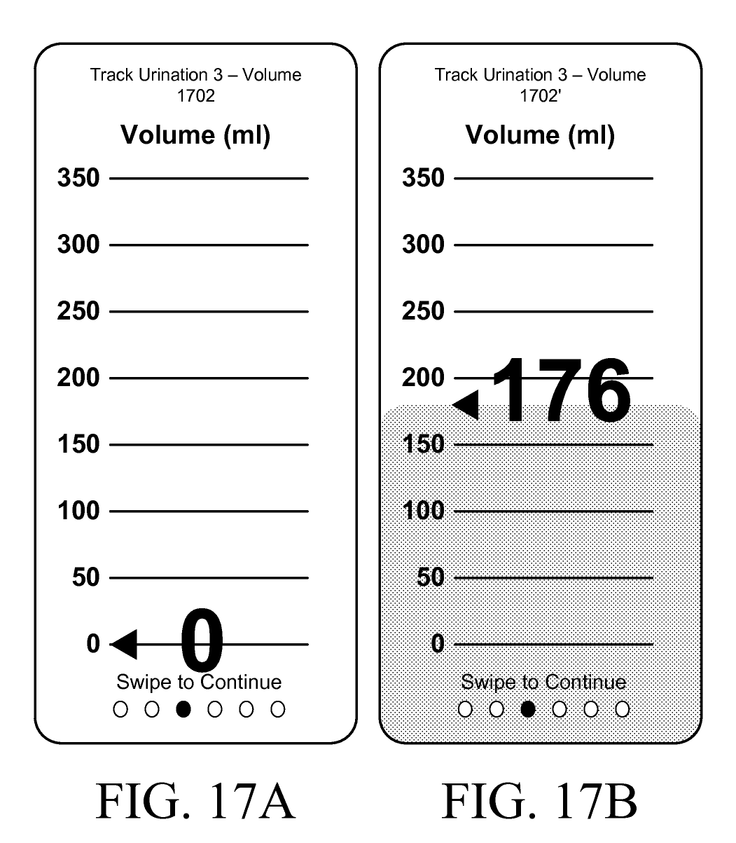

FIGS. 17A and 17B show example urination volume entry user interfaces 1702 and 1702′, respectively. FIG. 17A shows a starting urination volume user interface at a “0” ml level. FIG. 17B shows a urination volume user interface at an example level of 176 ml. Once the volume entry user interface screen has been cause to be displayed, a user can simply indicate via touch on the volume scale how much urine was output. A gesture on a touch screen (e.g., “left swipe”) can be used to move to the next step of tracking urination.

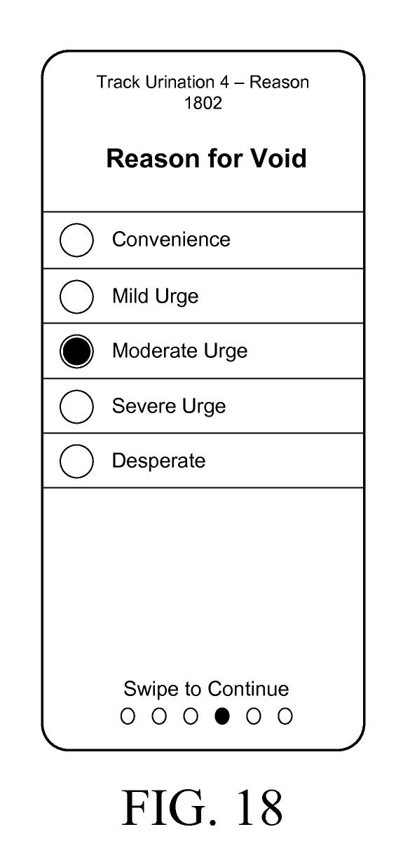

FIG. 18 shows a next urination tracking screen 1802 for capturing a reason for the void (e.g., convenience, mild urge, moderate urge, sever urge, desperate, etc.). A gesture on a touch screen (e.g., “left swipe”) can be used to move to the next step of tracking urination.

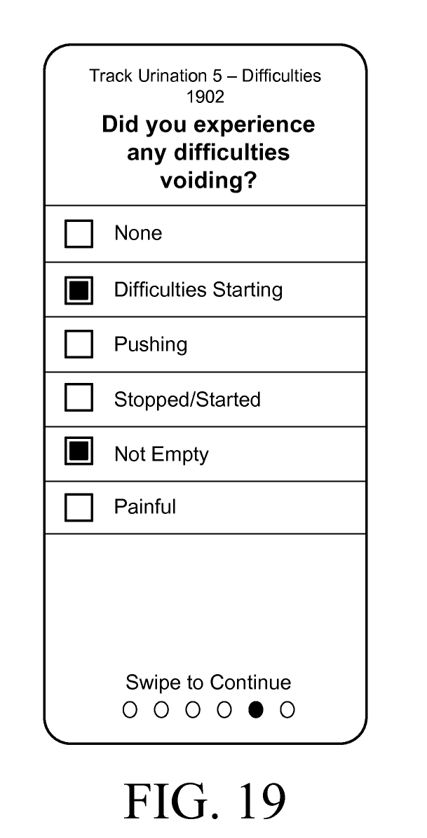

FIG. 19 shows a next urination tracking screen 1902 for capturing whether the user had any difficulties with the urination event being recorded. For example, the user can select one or more of none, difficulties starting, pushing, stopped/started, not empty, and painful. A gesture on a touch screen (e.g., “left swipe”) can be used to move to the next step of tracking urination.

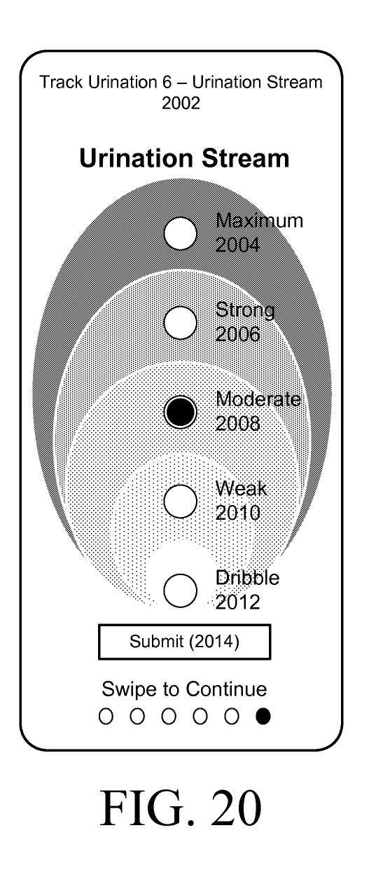

FIG. 20 shows a next urination tracking/recording user interface screen 2002 for capturing urination stream information. The user/patient can select from a urination stream of maximum 2004, strong 2006, moderate 2008, weak 2010 and dribble 212. Once the urination stream strength has been selected, the user can complete the urination tracking/recording by selecting submit 2014.

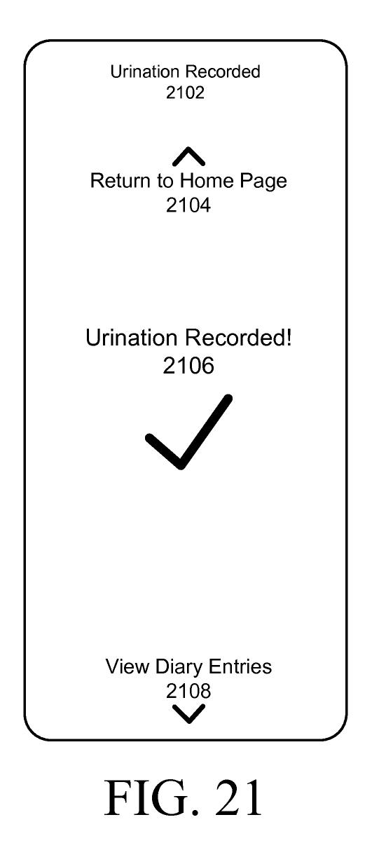

FIG. 21 shows a urination recorded screen 2102 having a return to home element 2104 that, when selected returns the user to the home screen, a urination recorded element 2106, and a view diary entries element 2108 that, when selected, causes diary entries to be displayed.

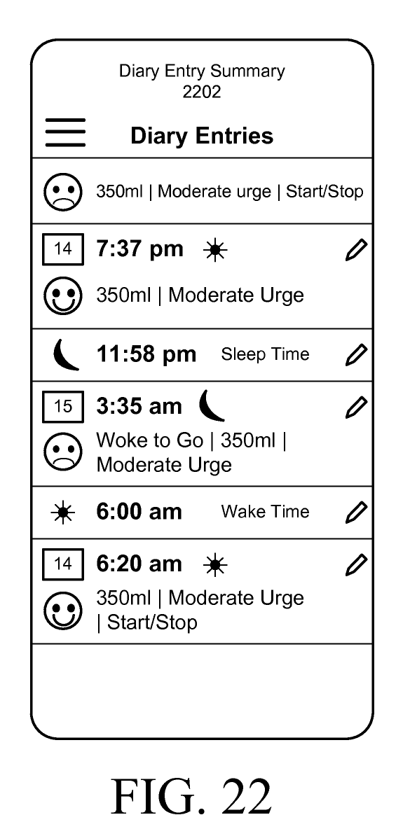

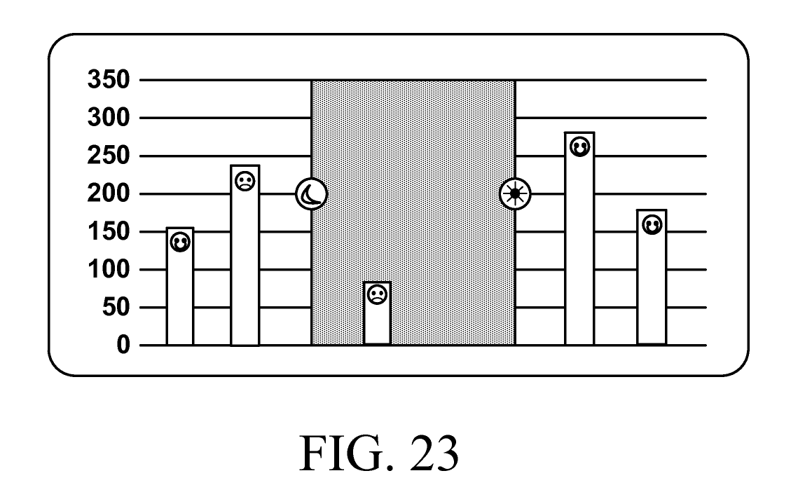

FIG. 22 shows an example diary entries screen. FIG. 23 shows an example horizontal format diaries entry screen. The diary entry screens shown in FIGS. 22 and 23 are examples of diary entry screens than can be caused to be displayed when a diary entries user interface element is selected.

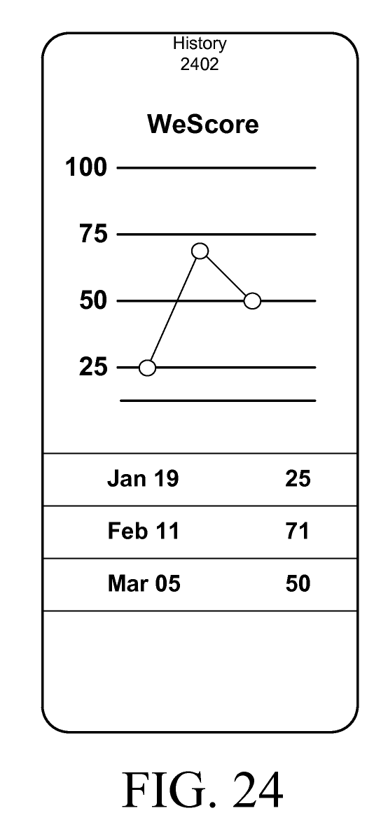

FIG. 24 shows an example score tracking screen showing the score (e.g., weScore) over a given range of time.

Once the patient completes a mobile application diary and questionnaire to generate a score (e.g., a WeScore™ as generated by a Symptelligence mobile application) on their mobile phone, the data (e.g., patient entered data, diary entries, questionnaire answers and/or scores) can be automatically sent to a HIPPA compliant secure server and can be accessible to a physician through the physician portal.

The physician can log into the physician portal by authenticating by entering his/her unique username and password (e.g., similar to that shown in FIG. 4). An initial screen (e.g., FIG. 5) can be displayed that shows a list of updates, patients, their activity and their scores (e.g., WeScore™). To view a patient's record, a physician can click on patient name.

In some implementations, scores (e.g., WeScore™) range from 0 to 100. Where, 0 means no symptoms and 100 indicates worst possible symptoms. There may be no cutoff between normal and abnormal, but there may be cutoffs for subscores. When a physician views the WeScore™, if the number is flashing red (or is otherwise visually altered), one or more components of the score are abnormal.

When a physician selects or “clicks” on the “LUTSS Scores” element to look at the total and individual scores, a screen is caused to be displayed that includes the total score and individual scores.

If the patient has significant voiding symptoms on the LUTSS, the bladder diary can be checked for difficulty voiding episodes and what types of difficulties were mentioned.

As can be understood from the above physician portal example, the mobile application medical diary and questionnaire can be used to capture patient medical information at one or more times, generate one or more scores based on the captured information and then provide the captured information and/or scores to a physician device at a later time for inclusion into a patient's electronic medical record and to provide the physician with patient symptom information upon which to base a diagnosis and/or treatment plan.

Diary entry information as discussed herein can include data entered by a patient (or derived from information entered by a patient) via a mobile device. the information can include information corresponding to one or more of the following: 24 hour voided volume, number of voids, urinary frequency, maximum voided volume (MVV), number of urgency episodes, number of incontinent episodes, number of voiding difficulty episodes, night-time total voided volume (e.g., NUV—nocturnal urine volume), number of night-time voids, primary nocturia voids—this is the number of times that a person is awakened from sleep by the urge to void, insomnia voids, nocturnal polyuria index and/or nocturia index.

It will be appreciated that the modules, processes, systems, and sections described above can be implemented in hardware, hardware programmed by software, software instructions stored on a nontransitory computer readable medium or a combination of the above. A system as described above, for example, can include a processor configured to execute a sequence of programmed instructions stored on a nontransitory computer readable medium. For example, the processor can include, but not be limited to, a personal computer or workstation or other such computing system that includes a processor, microprocessor, microcontroller device, or is comprised of control logic including integrated circuits such as, for example, an Application Specific Integrated Circuit (ASIC). The instructions can be compiled from source code instructions provided in accordance with a programming language such as Java, C, C++, C#.net, assembly or the like. The instructions can also comprise code and data objects provided in accordance with, for example, the Visual Basic™ language, or another structured or object-oriented programming language. The sequence of programmed instructions, or programmable logic device configuration software, and data associated therewith can be stored in a nontransitory computer-readable medium such as a computer memory or storage device which may be any suitable memory apparatus, such as, but not limited to ROM, PROM, EEPROM, RAM, flash memory, disk drive and the like.

Furthermore, the modules, processes systems, and sections can be implemented as a single processor or as a distributed processor. Further, it should be appreciated that the steps mentioned above may be performed on a single or distributed processor (single and/or multi-core, or cloud computing system). Also, the processes, system components, modules, and sub-modules described in the various figures of and for embodiments above may be distributed across multiple computers or systems or may be co-located in a single processor or system. Example structural embodiment alternatives suitable for implementing the modules, sections, systems, means, or processes described herein are provided below.

The modules, processors or systems described above can be implemented as a programmed general purpose computer, an electronic device programmed with microcode, a hard-wired analog logic circuit, software stored on a computer-readable medium or signal, an optical computing device, a networked system of electronic and/or optical devices, a special purpose computing device, an integrated circuit device, a semiconductor chip, and/or a software module or object stored on a computer-readable medium or signal, for example.

Embodiments of the method and system (or their sub-components or modules), may be implemented on a general-purpose computer, a special-purpose computer, a programmed microprocessor or microcontroller and peripheral integrated circuit element, an ASIC or other integrated circuit, a digital signal processor, a hardwired electronic or logic circuit such as a discrete element circuit, a programmed logic circuit such as a PLD, PLA, FPGA, PAL, or the like. In general, any processor capable of implementing the functions or steps described herein can be used to implement embodiments of the method, system, or a computer program product (software program stored on a nontransitory computer readable medium).

Furthermore, embodiments of the disclosed method, system, and computer program product (or software instructions stored on a nontransitory computer readable medium) may be readily implemented, fully or partially, in software using, for example, object or object-oriented software development environments that provide portable source code that can be used on a variety of computer platforms. Alternatively, embodiments of the disclosed method, system, and computer program product can be implemented partially or fully in hardware using, for example, standard logic circuits or a VLSI design. Other hardware or software can be used to implement embodiments depending on the speed and/or efficiency requirements of the systems, the particular function, and/or particular software or hardware system, microprocessor, or microcomputer being utilized. Embodiments of the method, system, and computer program product can be implemented in hardware and/or software using any known or later developed systems or structures, devices and/or software by those of ordinary skill in the applicable art from the function description provided herein and with a general basic knowledge of the software engineering and medical informatics arts.

Moreover, embodiments of the disclosed method, system, and computer readable media (or computer program product) can be implemented in software executed on a programmed general purpose computer, a special purpose computer, a microprocessor, or the like.

It is, therefore, apparent that there is provided, in accordance with the various embodiments disclosed herein, patient-centric medical information systems (and methods and computer readable media) including a mobile application and a physician portal.

While the disclosed subject matter has been described in conjunction with a number of implementations, it is evident that many alternatives, modifications and variations would be, or are, apparent to those of ordinary skill in the applicable arts. Accordingly, Applicants intend to embrace all such alternatives, modifications, equivalents and variations that are within the spirit and scope of the disclosed subject matter.

What is claimed is:

1. A method comprising:
registering, at one or more processors, a patient application installed on a mobile device;
receiving, at the one or more processors, at least a portion of a medical history of a patient, one or more medical diary entries and one or more patient responses to questions presented via the patient application;
generating at least one score based on the one or more medical diary entries and the one or more patient responses;
transferring, from the one or more processors to an external system, the medical history the medical diary entries, one or more patient responses and the at least one score.

2. The method of claim 1, further comprising:
providing for display on the external system, the medical history the medical diary entries, one or more patient responses and the at least one score.

3. The method of claim 2, wherein the external system includes a physician portal.

4. The method of claim 1, wherein the at least one score includes at least one score and at least one sub score.

5. A system comprising:
one or more processors coupled to a memory device having stored thereon software instruction that, when executed by the one or more processors, cause the processor sot perform operations including:
registering, at one or more processors, a patient application installed on a mobile device;
receiving, at the one or more processors, at least a portion of a medical history of a patient, one or more medical diary entries and one or more patient responses to questions presented via the patient application;
generating at least one score based on the one or more medical diary entries and the one or more patient responses;
transferring, from the one or more processors to an external system, the medical history the medical diary entries, one or more patient responses and the at least one score.

6. The system of claim 5, wherein the operations further comprise:
providing for display on the external system, the medical history the medical diary entries, one or more patient responses and the at least one score.

7. The system of claim 6, wherein the external system includes a physician portal.

8. The system of claim 5, wherein the at least one score includes at least one score and at least one sub score.

9. A nontransitory computer readable medium having stored thereon instructions that, when executed by a process, cause the processor to perform operations including:
registering, at one or more processors, a patient application installed on a mobile device;
receiving, at the one or more processors, at least a portion of a medical history of a patient, one or more medical diary entries and one or more patient responses to questions presented via the patient application;
generating at least one score based on the one or more medical diary entries and the one or more patient responses;
transferring, from the one or more processors to an external system, the medical history the medical diary entries, one or more patient responses and the at least one score.

10. The nontransitory computer readable medium of claim 9, wherein the operations further comprise:
providing for display on the external system, the medical history the medical diary entries, one or more patient responses and the at least one score.

11. The nontransitory computer readable medium of claim 10, wherein the external system includes a physician portal.

12. The nontransitory computer readable medium of claim 9, wherein the at least one score includes at least one score and at least one sub score.
